(19) United States
(12) Patent Application Publication
(10) Pub. No.: US 2025/0057626 A1
(43) Pub. Date: Feb. 20, 2025

(54) SYSTEM AND METHOD FOR ORIENTING A MEDICAL OBJECT ACCORDING TO A TARGET POSITIONING

(71) Applicant: Siemens Healthineers AG, Forchheim (DE)

(72) Inventor: Alois Regensburger, Poxdorf (DE)

(21) Appl. No.: 18/804,468

(22) Filed: Aug. 14, 2024

(30) Foreign Application Priority Data
Aug. 17, 2023 (DE) 10 2023 207 904.6

(51) Int. Cl. A61B 90/13; A61B 90/00; A61B 90/35

(52) U.S. Cl. CPC A61B 90/13; A61B 90/35; A61B 90/37; A61B 2090/376

(57) ABSTRACT

Systems and methods for providing support during an orienting of a medical object according to a target positioning. A light guidance device is configured to emit a predefined light distribution. The reflector element is configured to be fixedly mounted in a defined arrangement on the medical object or wherein the reflector element is integrated into the medical object in the defined arrangement. The reflector element is configured to reflect at least a part of the light distribution in a defined manner relative to the medical object when illuminated by the light distribution. The projection surface has a marker. The projection surface and the light guidance device are arranged in a defined positional relationship with respect to one another. The projection surface is illuminated by the reflected part of the light distribution. The reflected part of the light distribution illuminates the marker precisely when the medical object is oriented according to the target positioning.

### CROSS REFERENCE TO RELATED APPLICATIONS

This application claims the benefit of DE 10 2023 207 904.6, filed on Aug. 17, 2023, which is hereby incorporated by reference in its entirety.

### FIELD

Embodiments relate to a system for providing support during an orienting of a medical object according to a target positioning, to a medical object and to a method for orienting a medical object according to a target positioning.

### BACKGROUND

Within the context of minimally invasive interventions, for example bone interventions, for example vertebroplasty and/or kyphoplasty procedures, a bone trocar, a K-wire, a stiff needle, and/or a screw-like tool are often introduced as an interventional instrument into a bone structure of an examination subject, for example by hammering and/or drilling. Often, the interventional instrument needs to be guided to a target object by way of an entry site on the examination subject. For this purpose, it is often essential to monitor a positioning, for example an orientation and/or a position, of the interventional instrument relative to the examination subject. A fluoroscopy scan for intraprocedural navigation and/or guidance of the interventional instrument, for example by a C-arm X-ray device, is often used. No planning imaging and/or 3D imaging are performed preinterventionally in most cases.

With the aid of the fluoroscopic imaging it is possible, for example, to adjust a positioning of the interventional instrument and/or an angulation of the C-arm X-ray device as necessary. Alternatively or in addition, the interventional instrument, for example a longitudinal extension direction of the interventional instrument, may be detected in the fluoroscopy images and, by overlaying of a straight line, may be displayed as a graphical representation by a visualization unit. The superimposed straight line may be aligned onto the target object by iterative repositioning of the interventional instrument under fluoroscopy imaging. The known methods disadvantageously lead to an increased exposure of the examination subject to radiation. Furthermore, the methods are often time-consuming. The 2D imaging used has the disadvantage that an angulation of the C-arm must be changed at regular intervals in order to be able to rule out “off-plane” tilting of the trocar. By “off-plane” is meant a portion of the change in a direction vector and/or orientation vector of the shaft of the instrument that is collinear or parallel to the projection direction of the C-arm. This off-plane change of direction is not detectable or at best very difficult to detect in the case of thin, for example needle-or screw-like, instruments in a 2D X-ray projection. For example, during a progression view, a change in direction of the interventional instrument outside of the plane in the projection direction is to be identified that cannot be detected at the C-arm without changing to a second, for example orthogonal, progression view at a different angulation of the C-arm. An all too frequent change of angulation is extremely disadvantageous for a surgical workflow.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide for a medical object to be oriented relative to an examination subject in a time-efficient manner and with a low X-ray dose.

In a first aspect, a system is provided for support during an orienting of a medical object according to a target positioning. The system includes a light guidance device, a reflector element, and a projection surface. The light guidance device is configured to emit a predefined light distribution. The reflector element is configured to be fixedly mounted to the medical object in a defined arrangement. Alternatively, the reflector element is integrated into the medical object in the defined arrangement. Further, the reflector element, when illuminated by the light distribution, is configured to reflect at least a part of the light distribution in a defined manner relative to the medical object. The projection surface has a marker. The projection surface and the light guidance device are furthermore arranged in a defined positional relationship with respect to one another. In this arrangement, the projection surface may be illuminated by the reflected part of the light distribution. Furthermore, the reflected part of the light distribution illuminates the marker precisely when the medical object is oriented according to the target positioning.

The medical object may be a surgical instrument, for example a needle, for example a puncture needle, and/or a drill, and/or a diagnostic instrument, for example an endoscope, for example a laparoscope, and/or a catheter, and/or a trocar. The medical object may be configured at least partly, for example completely, as rigid and elongate, for example rod-and/or needle-shaped. The medical object, for example a distal section of the medical object, may be arranged on or in the examination subject in the operating state of the device. For example, the medical object may be arranged at least partly on or in the examination subject in the operating state of the device.

The light guidance device may include a light source, for example a laser light source, that is configured to emit the predefined light distribution. For this purpose, the light guidance device may for example include an optical diaphragm. The light guidance device may emit the light distribution in an operating state of the device.

The reflector element may be fixedly secured, for example arranged, in a defined positional relationship, for example in a defined spatial relative position and/or relative orientation and/or relative pose, on the medical object, for example on a proximal section of the medical object. For example, the reflector element may be releasably fixable to the medical object in the defined positional relationship. For this purpose, the reflector element and/or the medical object may include a fastening element, for example a clamping device and/or a plug-and-socket device and/or a magnetic holder. Alternatively, the reflector element may be integrated into the medical object, for example on a proximal section of the medical object, for example on a surface of the medical object. The reflector element may include an optical reflector, for example a mirror. The reflector may be configured as flat or curved, for example concave or convex. The reflector element may be arranged on the medical object in such a way that the reflector element, for example the reflector, may be illuminated by the light distribution.

The reflector element, for example the reflector, when illuminated by the light distribution, may be configured to reflect at least a part of the light distribution in a defined manner relative to the medical object. The arrangement of medical object and reflector element may be positioned in the operating state of the system in such a way that the defined light distribution illuminates the reflector element, for example the reflector. The light distribution may illuminate the reflector element in the operating state of the system at an angle of incidence relative to an axis of symmetry of the reflector. The reflector may be configured to reflect the at least one part of the predefined light distribution at an angle of reflection relative to the axis of symmetry. As a result of the defined arrangement of the reflector element relative to the medical object, the axis of symmetry of the reflector may have a defined positional relationship relative to the medical object, for example to a longitudinal extension axis of the medical object.

The system may further have a flat projection surface or a projection surface extending in a curved shape. The projection surface may for example include a surface, for example a housing surface, of a medical device, and/or a surface of the examination subject. The projection surface may have at least one marker. The at least one marker may be configured as a structural and/or anatomical element, for example an elevation or recess, and/or as a graphical element, for example an inscription, on the projection surface.

The projection surface, for example the at least one marker, and the light guidance device may be arranged in a defined positional relationship, for example in a defined spatial relative position and/or relative orientation and/or relative pose, with respect to one another. The reflected part of the light distribution illuminates the marker corresponding to the target positioning on the projection surface precisely when the medical object, for example together with the reflector element, is oriented according to the target positioning. The target positioning may include a spatial target position and/or target orientation and/or target pose of the medical object.

The system may provide the medical object to be oriented relative to the examination subject in a time-efficient manner and with a low X-ray dose. For example, an off-plane change in direction of the medical object may be visually detectable.

In an embodiment of the system, the system may further include a medical imaging device. The light guidance device and the projection surface may be arranged on the medical imaging device in a defined positional relationship with respect to one another and/or at least partly integrated into the medical imaging device.

The medical imaging device may include a medical X-ray device, for example a medical C-arm X-ray device and/or a cone-beam computed tomography system (CT, CBCT), and/or a computed tomography system (CT system) and/or a magnetic resonance tomography system (MRT system) and/or a positron emission tomography system (PET system) and/or an ultrasound device. The imaging device may be configured for acquiring and providing image data of the examination subject and of the medical object.

The light guidance device may be arranged on the medical imaging device, for example on a source or a detector, and/or integrated into the imaging device. Furthermore, the projection surface may be arranged on the medical imaging device and/or be at least partly integrated into the imaging device, for example in a housing of the imaging device.

This provides an inherent registration to exist between a coordinate system of the light guidance device and a coordinate system of the imaging device, as a result of which a combination of an image-guided orientation and an orientation by the projected light distribution may be realized particularly intuitively.

In an embodiment of the system, the medical imaging device may include an X-ray source and an X-ray detector that are mounted so as to be movable. The projection surface may be a surface of the X-ray detector facing toward the X-ray source.

The imaging device, for example the medical X-ray device, may include the X-ray source and the X-ray detector in a defined arrangement with respect to one another, for example on a C-arm. The defined arrangement of X-ray source and X-ray detector may be mounted so as to be movable, for example rotatable and/or capable of translatory motion.

The X-ray detector may have an X-ray-sensitive surface for detecting X-ray beams, for example for detecting X-ray radiation emitted by the X-ray source. The projection surface may be arranged on the same side, for example area and/or surface, of the X-ray detector as the X-ray-sensitive surface. For example, the projection surface may be a housing surface of the X-ray detector that is arranged along a beam path from the X-ray source to the X-ray detector ahead of the X-ray-sensitive surface of the X-ray detector. For example, the projection surface may be a surface facing toward the X-ray source, for example a housing surface, of the X-ray detector.

The embodiment may provide an off-plane change in the direction of the medical object to be detected without a change in angulation, for example without a change in a projection direction between X-ray source and X-ray detector. In the event of an, for example visually, identified off-plane change in direction, this may lead to an acquisition with rotated angulation, for example projection direction, becoming necessary. Alternatively, the off-plane change in direction may be identified directly in image data acquired by the X-ray device at the original angulation.

In an embodiment of the system, the X-ray source and the X-ray detector may be movably mounted around a common isocenter. Furthermore, the predefined light distribution may illuminate the isocenter.

The X-ray source and the X-ray detector, for example the defined arrangement of X-ray source and X-ray detector, may be movably, for example rotatably, mounted around the isocenter, for example around a center of rotation. The light guidance device may further be configured to emit the defined light distribution in such a way that the isocenter of the medical imaging device may be illuminated by the light distribution. For example, the light guidance device may emit the light distribution in an operating state of the system in such a way that the isocenter would be able to be illuminated by the light distribution in the case of uncovered lighting.

At least one section of the medical object is arranged at the isocenter. This may provide the section of the medical object to be imaged from multiple projection directions, for example angulations, by the X-ray device. The section of the medical object arranged in the isocenter may further be imaged in a center of the 2D X-ray image that may be acquired by the X-ray device. This provides a spatial distance to be determined between the medical object, for example the section of the medical object arranged in the isocenter, and the X-ray detector and/or the X-ray source. As a result, for example, an improved assumption may be made about a position of a center of rotation of a tilting of the medical object when being oriented according to the target positioning.

In an embodiment of the system, the predefined light distribution may project a geometric shape.

The light guidance device may be configured to project a light pattern having the geometric shape by the predefined light distribution. The reflector element, when illuminated by the light distribution, may reflect at least a part of the projected light pattern in a defined manner relative to the medical object. The reflector element may project the reflected part of the light pattern onto the projection surface. The geometric shape may include, for example, a line and/or a point and/or a point cloud and/or a cross and/or a circle and/or a polygon.

This may provide an improved identification as to whether the reflected part of the light distribution illuminates the specified marker. This further allows an improvement in identifying whether the medical object is oriented according to the target positioning.

In an embodiment of the system, the light guidance device may be configured to emit a further predefined light distribution for illuminating the projection surface, which light distribution projects a further light pattern onto the projection surface in an operating state of the system. At the same time, the projection of the further light pattern on the projection surface may at least partly, for example completely, form the marker.

The further predefined light distribution may for example possess all the properties and features of the predefined light distribution, and vice versa. The further light pattern may include a further geometric shape, for example a line and/or a point and/or a point cloud and/or a cross and/or a circle and/or a polygon. The projection of the light pattern, for example of the geometric shape, on the projection surface may partly, for example in addition to a marker configured as a structural and/or anatomical and/or graphical element, or completely form the marker.

The embodiment may provide a flexible adjustment of a positioning and/or appearance, for example shape, of the marker on the projection surface. As a result, a flexible orientation of the medical object may be realized according to the respective specified target positioning.

In an embodiment of the system, the projection surface may have a plurality of markers for a respective potential target positioning of the medical object. This provides the reflected part of the light distribution to illuminate the corresponding marker precisely when the medical object is oriented according to the respective target positioning.

The plurality of markers may be configured differently or similarly. Further, the plurality of markers may each possess an optically distinguishable property, for example a color coding and/or a black-and-white coding and/or a surface composition, for example a reflectivity and/or fluorescence and/or contour. The plurality of markers may be uniquely identifiable on the basis of the optically distinguishable property. Each of the markers may correspond in each case to precisely one potential, for example predefined, target positioning, for example target position and/or target orientation and/or target pose, of the medical object. The correspondence consists in the fact that the respective marker of the plurality of markers is illuminated by the reflected part of the light distribution precisely when the medical object is oriented according to the respective target positioning.

The embodiment may allow a flexible orientation of the medical object according to the specified target positioning in each case.

In an embodiment of the system, the plurality of markers may form a scale.

The plurality of markers may be disposed in a defined arrangement on the projection surface, for example equidistantly or at different distances from one another. Furthermore, the plurality of markers may have a coding, for example a color coding, and/or a legend, for example angle specifications. If the markers are configured as straight lines, the markers may be arranged parallel to one another. Furthermore, the plurality of markers may have a reference marker relative to which the remaining markers form a scale of variations.

The embodiment may provide an intuitive orientation of the medical object according to the respective target positioning and a qualitative and/or quantitative detection of a deviation relative to a reference positioning, for example corresponding to the reference marker.

In an embodiment of the system, the reflector element may be configured to be moved along a longitudinal extension direction of the medical object and be releasably secured to the medical object in the defined arrangement.

The reflector element may be capable of translatory movement along the longitudinal extension direction of the medical object and/or rotatable around the longitudinal extension direction. In addition, the reflector element and/or the medical object may include a locking element that provides for the reflector element to be releasably secured to the medical object in the defined arrangement.

This provides for a simple, for example subsequent, arrangement of the reflector element, tailored to the particular situation, on the medical object to be realized in the defined arrangement.

Embodiments provide a second aspect to a medical device including a medical object and a reflector element. The reflector element is disposed in a defined arrangement on the medical object or is integrated into the medical object in the defined arrangement. When illuminated by a light distribution, the reflector element is configured to reflect at least a part of the light distribution in a defined manner relative to the medical object.

The embodiments of the medical object substantially correspond to the embodiments of the system. Features, advantages, or alternative embodiments mentioned in this context may equally be applied also to the other claimed subject matters, and vice versa.

In an embodiment of the medical device, the medical object may include a distal and a proximal section. The distal section may be configured to be at least partly arranged in an examination subject. Furthermore, when the distal section is arranged inside the examination subject, the proximal section may be configured to be arranged outside of the examination subject. The reflector element may additionally be arranged on the proximal section.

The proximal section may include a spatial portion of the medical object that is arranged facing away from the examination subject, for example facing toward a member of the medical operating staff to allow manipulation of the medical object, for example along a longitudinal extension direction of the medical object. Further, the distal section may include a spatial portion of the medical object that is arranged facing toward the examination subject, for example facing away from a member of the medical operating staff to allow manipulation of the medical object, for example along a longitudinal extension direction of the medical object.

In an embodiment of the medical device, the reflector element may be configured to be moved along a longitudinal extension direction of the medical object and to be releasably secured to the medical object in the defined arrangement.

Embodiments provide a third aspect to a method for orienting a medical object according to a target positioning. In a first step a), a predefined light distribution is emitted by a light guidance device. The light distribution illuminates a reflector element that is fixedly mounted in a defined arrangement on the medical object or is integrated into the medical object in the defined arrangement. The reflector element reflects at least a part of the light distribution in a defined manner relative to the medical object. In a second step b), a check is conducted to determine whether a specified marker on a projection surface is illuminated by the reflected part of the light distribution. The projection surface and the light guidance device are arranged in a defined positional relationship with respect to one another. If the check proves negative, the medical object is repositioned and step b) is repeated.

The check in step b) may be performed manually, for example by a member of the medical operating staff, or automatically, for example by an optical sensor and/or a camera. If it is confirmed during the check in step b) that the specified marker on the projection surface is illuminated by the reflected part of the light distribution, then the method may end at this point. If it is found during the check in step b) that the specified marker on the projection surface is not illuminated by the reflected part of the light distribution, then the medical object may be repositioned, for example rotated and/or translated, and step b) may be repeated. The relative positioning of the reflector element with respect to the projection surface and the light guidance device may change as a result of the repositioning of the medical object. The repositioning of the medical object and step b) may be performed repeatedly until the specified marker on the projection surface is illuminated by the reflected part of the light distribution.

The embodiments of the method substantially correspond to the advantages of the system. Features, advantages, or alternative embodiments mentioned in this context may equally be applied also to the other claimed subject matters, and vice versa.

In an embodiment of the method, the predefined light distribution may project a geometric shape.

In an embodiment of the method, the light guidance device may emit a further predefined light distribution for illuminating the projection surface, which light distribution projects a further light pattern onto the projection surface. Furthermore, the projection of the further light pattern on the projection surface may at least partly, for example completely, form the marker.

### BRIEF DESCRIPTION OF THE FIGURES

FIGS. 1, 2, and 3 depict schematic views of different embodiments of a system.

FIGS. 4 and 5 depict schematic views of different embodiments of markers on a projection surface.

FIG. 6 depicts a schematic view of a medical object according to an embodiment.

FIGS. 7 and 8 depict schematic views of different embodiments of a method for orienting a medical object according to a target positioning.

### DETAILED DESCRIPTION

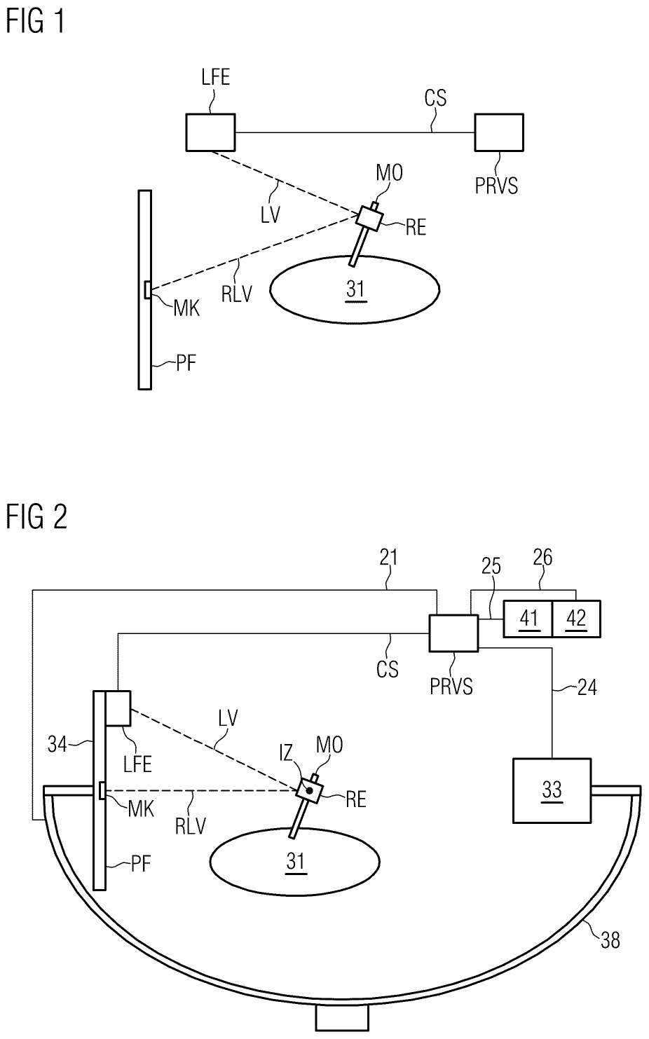

FIG. 1 depicts a schematic view of an embodiment of a system for providing support during an orienting of a medical object MO according to a target positioning. The system may include a light guidance device LFE, a reflector element RE and a projection surface PF. The light guidance device LFE may be configured to emit a predefined light distribution LV. The reflector element RE may be configured to be fixedly secured in a defined arrangement on the medical object MO. Alternatively, the reflector element RE may be integrated into the medical object MO in the defined positional relationship. Furthermore, the reflector element RE may be configured to reflect at least a part of the light distribution RLV in a defined manner relative to the medical object MO when illuminated by the light distribution LV. The projection surface PF may have a marker MK. The projection surface PF and the light guidance device LFE may further be arranged in a defined positional relationship with respect to one another. The projection surface PF may be illuminated by the reflected part of the light distribution RLV. The reflected part of the light distribution RLV may illuminate the marker MK precisely when the medical object MO is oriented according to the target positioning.

The medical object MO, for example a distal section of the medical object MO, may be arranged at least partly on or in an examination subject 31 in an operating state of the system. The target positioning may specify a spatial target position and/or target orientation and/or target pose of the medical object MO relative to the examination subject 31.

The predefined light distribution LV may project a geometric shape.

The reflector element RE may be configured to be moved along a

longitudinal extension direction of the medical object MO and to be releasably secured to the medical object MO in the defined arrangement.

FIG. 2 depicts a schematic view of an embodiment of a system for providing support during an orienting of a medical object MO according to a target positioning. The system may further include a medical imaging device, for example a medical C-arm X-ray device. The X-ray device may include an X-ray source 33 and an X-ray detector 34 that are mounted in a defined arrangement on a common C-arm 38 so as to be movable around an isocenter IZ. The light guidance device LFE and the projection surface PF may be arranged on the medical imaging device in a defined positional relationship with respect to one another and/or be at least partly integrated into the medical imaging device. For example, the light guidance device LFE may be arranged on the X-ray detector 34. Further, the projection surface PF may be a surface of the X-ray detector 34 facing toward the X-ray source 33. The predefined light distribution LV may furthermore illuminate the isocenter IZ.

In order to acquire image data of the examination subject 31, the provisioning unit PRVS may send a signal 24 to the X-ray source 33. The X-ray source 33 may thereupon emit an X-ray beam. When the X-ray beam, following an interaction with the examination subject 31, impinges on a surface of the detector 34, the detector 34 may send a signal 21 to the provisioning unit PRVS. The provisioning unit PRVS may acquire the image dataset on the basis of the signal 21.

The system may further include an input unit 42 and a visualization unit 41. The input unit 42 may include, for example, a keyboard and/or a pointing device and/or a recognition unit, for example a voice recognition unit and/or a gesture recognition unit. The visualization unit 41 may further include a monitor and/or a display and/or a projector, for example. The input unit 42 may preferably be integrated into the visualization unit 41, for example in the case of a capacitive and/or resistive input display. The input unit 42 may be configured for registering a user input. The input unit 42 may for example send a signal 26 to the provisioning unit PRVS. The provisioning unit PRVS may be configured to control the emission of the light distribution LV, for example on the basis of the user input, for example by a signal CS. The provisioning unit PRVS may be further configured to control the X-ray device for the acquisition of the image data on the basis of the user input.

The visualization unit 41 may be configured to display a graphical representation of the target positioning and/or of a workflow notification and/or of the medical image data. The provisioning unit PRVS may send a signal 25 to the visualization unit 41 for this purpose.

FIG. 3 depicts a schematic view of an embodiment of a system for providing support during an orienting of a medical object MO according to a target positioning. The light guidance device LFE may be configured to emit a further predefined light distribution FLV for illuminating the projection surface PF, which further light distribution projects a further light pattern onto the projection surface PF in an operating state of the system. The projection of the further light pattern on the projection surface may at least partly, for example completely, form the marker MK.

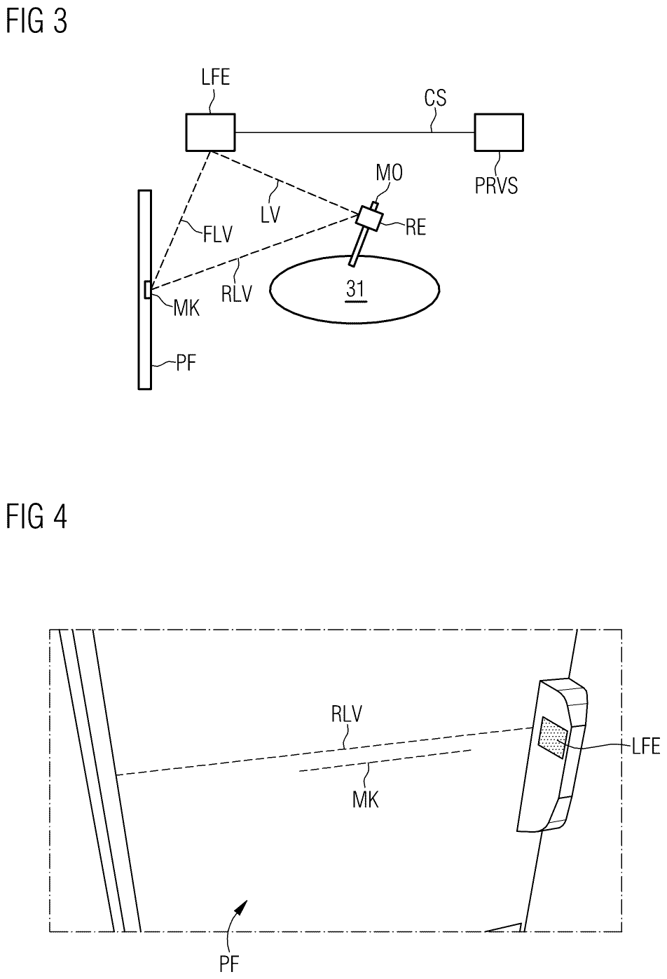

FIG. 4 depicts a schematic view of a marker MK formed by the further light pattern FLV projected onto the projection surface PF.

FIG. 5 depicts a schematic view of a plurality of markers MKR, MK1 and MK2 on the projection surface PF for a respective potential target positioning of the medical object MO. The reflected part of the light distribution RLV may illuminate the corresponding marker precisely when the medical object MO is oriented according to the respective target positioning. As depicted in FIG. 5, the light guidance device LFE may be arranged on the projection surface PF, for example on the X-ray detector 34. The light guidance device LFE may include a deflection element AE, for example a mirror, that is configured to deflect the predefined light distribution LV in a defined manner.

The plurality of markers MKR, MK1 and MK2 may be configured

differently or similarly. Further, the plurality of markers MKR, MK1 and MK2 may each possess an optically distinguishable property, for example a color coding. The plurality of markers MKR, MK1 and MK2 may be uniquely identifiable on the basis of the optically distinguishable property. Each of the markers MKR, MK1 and MK2 may correspond in each case to precisely one potential, for example predefined, target positioning of the medical object MO. The correspondence consists in the fact that the respective marker of the plurality of markers MKR, MK1 and MK2 is illuminated by the reflected part of the light distribution RLV precisely when the medical object MO is oriented according to the respective target positioning. The plurality of markers MKR, MK1 and MK2 may be disposed in a defined arrangement on the projection surface PF, for example equidistantly or at different distances from one another. If the markers MKR, MK1 and MK2 are configured as straight lines, they may be arranged parallel to one another. Furthermore, the plurality of markers may include a reference marker MKR relative to which the remaining markers MK1 and MK2 form a scale of variations.

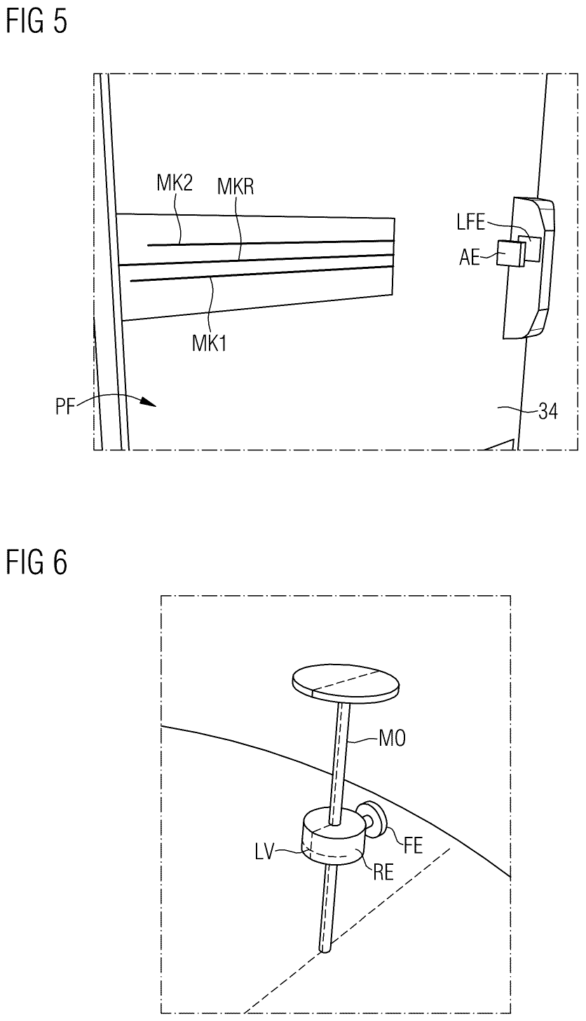

FIG. 6 depicts a schematic view of a medical device including a medical object MO and the reflector element RE. The reflector element RE may be integrated into the medical object in a defined arrangement or be disposed on the medical object MO in the defined arrangement. Further, the reflector element RE, when illuminated by a light distribution LV, may be configured to reflect at least a part of the light distribution in a defined manner relative to the medical object MO. The medical object may include a distal and a proximal section. The distal section may be configured to be arranged at least partly in an examination subject 31. Furthermore, the proximal section may be configured to be arranged outside of the examination subject 31 when the distal section is arranged inside the examination subject 31. The reflection element RE may be arranged on the proximal section. For example, the reflector element RE may be configured to be moved along a longitudinal extension direction of the medical object MO and to be releasably secured to the medical object MO in the defined arrangement, for example by a locking element FE.

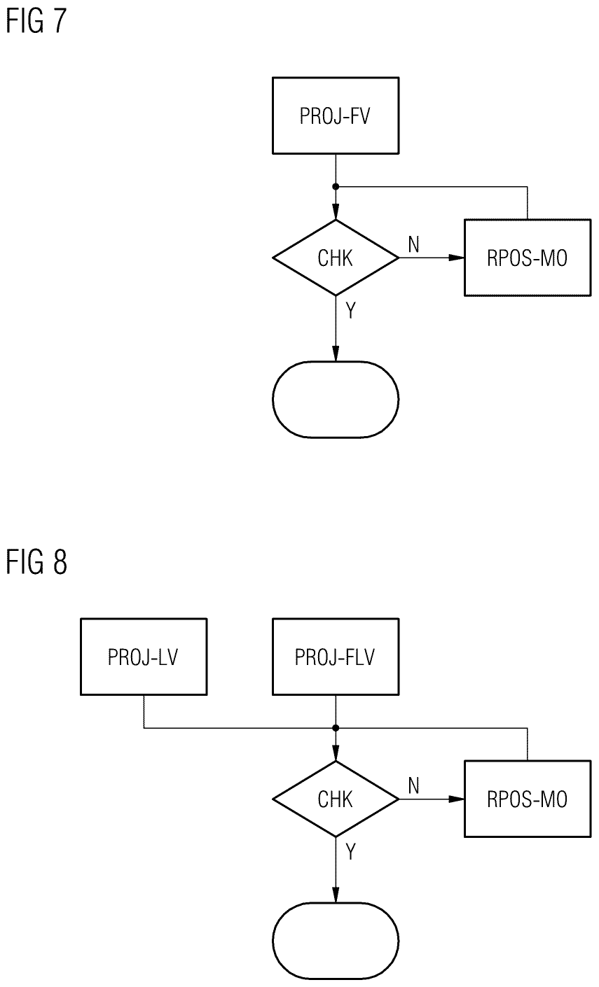

FIG. 7 depicts a schematic view of an embodiment of a method for orienting a medical object MO according to a target positioning. In a first step, the predefined light distribution LV may be emitted PROJ-LV by the light guidance device LFE. The light distribution LV may illuminate the reflector element RE, that is fixedly mounted in a defined arrangement on the medical object MO or integrated into the medical object MO in the defined arrangement. Furthermore, the reflector element RE may reflect at least a part of the light distribution RLV in a defined manner relative to the medical object MO. In a further step, it may be checked CHK whether a specified marker on the projection surface PF is illuminated by the reflected part of the light distribution RLV. The projection surface PF and the light guidance device LFE may be arranged in a defined positional relationship with respect to one another. If the check proves negative N, the medical object MO may be repositioned RPOS-MO and the check CHK repeated.

The schematic views contained in the described figures are in no way representative of scale or proportions.

The methods described in detail in the foregoing, as well as the illustrated devices, are merely embodiments that may be modified in the most diverse ways by the person skilled in the art without leaving the scope of the invention. Furthermore, the use of the indefinite articles “a” or “an” does not exclude the possibility that the features in question may also be present more than once. Similarly, the terms “unit” and “element” do not rule out the possibility that the components in question consist of a plurality of cooperating subcomponents that if necessary may also be distributed in space.

In the context of the present application, the expression “based on” may be understood for example in the sense of the term “using”. For example, a formulation according to which a first feature is generated (alternatively: ascertained, determined, etc.) based on a second feature does not rule out the possibility that the first feature may be generated (alternatively: ascertained, determined, etc.) based on a third feature.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A system for providing support during an orienting of a medical object according to a target positioning, the system comprising:
a light guidance device configured to emit a predefined light distribution;
a reflector element configured to be fixedly mounted in a defined arrangement on the medical object or wherein the reflector element is integrated into the medical object in the defined arrangement, wherein the reflector element, when illuminated by the light distribution, is configured to reflect at least a part of the light distribution in a defined manner relative to the medical object; and
a projection surface comprising at least a marker;
wherein the projection surface and the light guidance device are arranged in a defined positional relationship with respect to one another, wherein the projection surface is illuminated by the reflected part of the light distribution,
wherein the reflected part of the light distribution illuminates the marker precisely when the medical object is oriented according to the target positioning.

2. The system of claim 1, further comprising:
a medical imaging device, wherein the light guidance device and the projection surface are arranged on the medical imaging device in a defined positional relationship with respect to one another and/or are integrated at least partly into the medical imaging device.

3. The system of claim 2, wherein the medical imaging device comprises an X-ray source and an X-ray detector that are mounted so as to be movable, wherein the projection surface is a surface of the X-ray detector facing toward the X-ray source.

4. The system of claim 3, wherein the X-ray source and the X-ray detector are mounted so as to be movable around a common isocenter, wherein the predefined light distribution illuminates the isocenter.

5. The system of claim 1, wherein the predefined light distribution projects a geometric shape.

6. The system of claim 1, wherein the light guidance device is configured to emit a further predefined light distribution for illuminating the projection surface, that, in an operating state of the system, projects a further light pattern onto the projection surface, wherein the projection of the further light pattern on the projection surface at least partly forms the marker.

7. The system of claim 1, wherein the projection surface has a plurality of markers for a respective potential target positioning of the medical object, wherein the reflected part of the light distribution illuminates a corresponding marker precisely when the medical object is oriented according to the respective target positioning.

8. The system of claim 7, wherein the plurality of markers form a scale.

9. The system of claim 1, wherein the reflector element is configured to be moved along a longitudinal extension direction of the medical object and to be releasably secured to the medical object in the defined arrangement.

10. A medical device comprising:
a medical object; and
a reflector element, wherein the reflector element is disposed in a defined arrangement on the medical object or is integrated into the medical object in the defined arrangement, wherein the reflector element, when illuminated by a light distribution, is configured to reflect at least a part of the light distribution in a defined manner relative to the medical object.

11. The medical device of claim 10, wherein the medical object comprises a distal and a proximal section, wherein the distal section is configured to be arranged at least partly in an examination subject, wherein the proximal section is configured to be arranged outside of the examination subject when the distal section is arranged inside the examination subject, wherein the reflector element is arranged on the proximal section.

12. The medical device of claim 11, wherein the reflector element is configured to be moved along a longitudinal extension direction of the medical object and to be releasably secured to the medical object in the defined arrangement.

13. A method for orienting a medical object according to a target positioning, the method comprising:
emitting a predefined light distribution by a light guidance device, wherein the light distribution illuminates a reflector element that is fixedly mounted in a defined arrangement on the medical object or is integrated into the medical object in the defined arrangement, wherein the reflector element reflects at least a part of the light distribution in a defined manner relative to the medical object; and
checking whether a specified marker on a projection surface is illuminated by the reflected part of the light distribution, wherein the projection surface and the light guidance device are arranged in a defined positional relationship with respect to one another, wherein the medical object is repositioned and checking is repeated if the check proves negative.

14. The method of claim 13, wherein the predefined light distribution projects a geometric shape.

15. The method of claim 14, wherein the light guidance device emits a further predefined light distribution for illuminating the projection surface, which further light distribution projects a further light pattern onto the projection surface, wherein the projection of the further light pattern on the projection surface at least partly forms the specified marker.

16. The method of claim 15, wherein the projection of the further light pattern on the projection surface completely forms the specified marker.
